SEQUENCE LISTING (8 sequences)

SEQ ID NO: 1 - DNA, 1008 bp, Saccharomyces cerevisiae
  CDS (1)..(1008): TAL1
atgtctgaaccagctcaaaagaaacaaaaggttgctaacaactctctagaacaattgaaagcctccggcactgtcgttgttgccgacactggtgatttcggctctattgccaagtttcaacctcaagactccacaactaacccatcattgatcttggctgctgccaagcaaccaacttacgccaagttgatcgatgttgccgtggaatacggtaagaagcatggtaagaccaccgaagaacaagtcgaaaatgctgtggacagattgttagtcgaattcggtaaggagatcttaaagattgttccaggcagagtctccaccgaagttgatgctagattgtcttttgacactcaagctaccattgaaaaggctagacatatcattaaattgtttgaacaagaaggtgtctccaaggaaagagtccttattaaaattgcttccacttgggaaggtattcaagctgccaaagaattggaagaaaaggacggtatccactgtaatttgactctattattctccttcgttcaagcagttgcctgtgccgaggcccaagttactttgatttccccatttgttggtagaattctagactggtacaaatccagcactggtaaagattacaagggtgaagccgacccaggtgttatttccgtcaagaaaatctacaactactacaagaagtacggttacaagactattgttatgggtgcttctttcagaagcactgacgaaatcaaaaacttggctggtgttgactatctaacaatttctccagctttattggacaagttgatgaacagtactgaacctttcccaagagttttggaccctgtctccgctaagaaggaagccggcgacaagatttcttacatcagcgacgaatctaaattcagattcgacttgaatgaagacgctatggccactgaaaaattgtccgaaggtatcagaaaattctctgccgatattgttactctattcgacttgattgaaaagaaagttaccgcttaa

SEQ ID NO: 2 - protein, 335 aa, Saccharomyces cerevisiae
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 3 - DNA, 33 bp, Artificial
  Forward primer
atcaggactagtatgtctgaaccagctcaaaag

SEQ ID NO: 4 - DNA, 37 bp, Artificial
  Reverse primer
aatcgcggatccttaagcggtaactttcttttcaatc

SEQ ID NO: 5 - DNA, 2043 bp, Saccharomyces cerevisiae
  CDS (1)..(2043): TKL1
atgactcaattcactgacattgataagctagccgtctccaccataagaattttggctgtggacaccgtatccaaggccaactcaggtcacccaggtgctccattgggtatggcaccagctgcacacgttctatggagtcaaatgcgcatgaacccaaccaacccagactggatcaacagagatagatttgtcttgtctaacggtcacgcggtcgctttgttgtattctatgctacatttgactggttacgatctgtctattgaagacttgaaacagttcagacagttgggttccagaacaccaggtcatcctgaatttgagttgccaggtgttgaagttactaccggtccattaggtcaaggtatctccaacgctgttggtatggccatggctcaagctaacctggctgccacttacaacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattctataagggtgacaagctaatttctcctttgaaaaaagctttctaa

SEQ ID NO: 6 - protein, 680 aa, Saccharomyces cerevisiae
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 7 - DNA, 32 bp, Artificial
  Forward primer
acgcgtcgacatgactcaattcactgacattg

SEQ ID NO: 8 - DNA, 35 bp, Artificial
  Reverse primer
atcaggactagtttagaaagcttttttcaaaggag

(19) United States
(12) Patent Application Publication
(10) Pub. No.: US 2016/0017379 A1
(43) Pub. Date: Jan. 21, 2016

(54) Process For Production Of Ethanol From Biomass

(71) Applicant: NATIONAL UNIVERSITY CORPORATION KOBE UNIVERSITY, Hyogo (JP)

(72) Inventors: Akihiko Kondo, Hyogo (JP); Tomohisa Hasunuma, Hyogo (JP); Tomoya Sanda, Hyogo (JP)

(73) Assignee: NATIONAL UNIVERSITY CORPORATION KOBE UNIVERSITY, Hyogo (JP)

(21) Appl. No.: 14/873,718

(22) Filed: Oct. 2, 2015

Related U.S. Application Data
(62) Division of application No. 13/512,047, filed on May 25, 2012.

(30) Foreign Application Priority Data
Nov. 30, 2009 (JP) 2009-272502

(51) Int. Cl. C12P 7/10; C12N 9/10

(52) U.S. Cl. CPC C12P 7/10; C12N 9/1022; C12Y 202/01001; C12Y 202/01002

(57) ABSTRACT

An object of the present invention is to provide a method for producing ethanol efficiently even in the presence of a fermentation inhibitor in a saccharified biomass. The present invention provides a method for producing ethanol from biomass, comprising: culturing a transformed xylose-utilizing yeast to overexpress the gene for at least one pentose phosphate pathway metabolic enzyme, with a saccharified biomass.

### TECHNICAL FIELD

The present invention relates to a method for producing ethanol from biomass.

### BACKGROUND ART

With a concern about depletion for fossil fuels, alternative fuels are now being developed. In particular, bioethanol derived from biomass is focused because biomass is a renewable resource which occurs in great abundance on earth, and can be used without increasing carbon dioxide in the atmosphere (carbon neutral) to contribute to prevention of global warming.

However, mainly corn and sugar cane are used as raw materials to produce bioethanol, which causes competition with food. Therefore, it is desired in the future to produce bioethanol using lignocellulose-based biomass, such as rice straw, straw, and wood scrap, as a raw material to avoid the competition with food.

Lignocellulose-based biomass is composed mainly of three components, cellulose, hemicellulose, and lignin. Among these, cellulose can be converted to glucose by saccharification, and then used in ethanol fermentation by a glucose-utilizing yeast such as Saccharomyces cerevisiae or the like. In contrast, hemicellulose can be converted to a pentose such as xylose or arabinose by saccharification, but is hardly used in ethanol production by fermentation in that naturally-occuring yeasts have a very poor ability to utilize xylose or arabinose.

Accordingly, for xylose utilization, an yeast has been genetically engineered to overexpress xylose reductase (XR) and xylitol dehydrogenase (XDH) derived from the yeast Pichia stipitis and the xylulokinase (XK) derived from the yeast Saccharomyces cerevisiae by introducing the genes for these enzymes (Non-Patent Documents 1 and 2). In addition, a yeast that allows ethanol fermentation from xylose has been made by introducing into a gene genes for xylose isomerase (XI) derived from anaerobic fungus Piromyces or Orpinomyces and XK derived from the yeast Saccharomyces cerevisiae to express them (Non-Patent Document 3).

Thus, ethanol fermentation from xylose has become possible. However, there are several problems with developing ethanol fermentation from xylose to an industrial scale, including, for example, a lower consumption rate, a lower ethanol production rate, and a lower ethanol yield with xylose than with glucose; and the presence of fermentation inhibitors in a saccharified solution, which is the problem to be mostly solved for putting ethanol production from cellulose-based biomass into practical use.

Cellulose-based biomass can be degraded (saccharified) to C6 sugar such as glucose, or C5 sugar such as xylose or arabinose using the process such as enzymatic treatment, treatment with diluted sulfuric acid or hydrothermal treatment. According to enzymatic treatment, enzymes are required in a large variety and amount, which causes the problem of cost with the development to an industrial scale; while according to treatment with diluted sulfuric acid or hydrothermal treatment, several overdegraded products (by-products) may occur, including weak acids such as acetic acid and formic acid; furan compounds such as furfural and hydroxymethylfurfural (HMF); and phenols including vanillin, and it has been known that such by-products are fermentation inhibitors which greatly inhibits ethanol fermentation from xylose (Non-Patent Documents 4 to 6). Therefore, a yeast that is tolerant to overdegraded products of biomass, or a yeast that is capable of efficient ethanol fermentation even in the presence of such fermentation inhibitors is desired so that cost-effective procedures, treatment with diluted sulfuric acid or hydrothermal treatment can be used to put ethanol fermentation from biomass into practical use.

Heretofore, the influence of fermentation inhibitors on yeasts has been investigated (Non-Patent Documents 4 to 6). It has been found that furfural has a great influence on the survival, growth rate, budding, ethanol yield, biomass yield, and enzyme activity in yeasts. It has been found that HMF causes accumulation of lipids, reduces the protein content, and inhibits alcohol dehydrogenase, aldehyde dehydrogenase, and pyruvate dehydrogenase in yeast cells. Research has been carried out using screening of disruption strains or transcriptional analysis to search for a gene tolerant to furfural or HMF (Non-Patent Documents 7 and 8).

Meanwhile, it was thought that weak acids such as acetic acid and formic acid would affect the pH in yeast cell, in other words, weak acids would occur in the medium in an undissociated form, and the undissociated weak acid would penetrate the cell membrane of yeast and enter the cytosol of the yeast with around neutral pH, and then become dissociated into an anion and a proton to cause pH decrease in the cell of the yeast (Non-Patent Document 4). Then, the pH decrease in the cell would activate ATPase to maintain homeostasis, so requiring ATP. Under anaerobic conditions, ATP is regenerated through ethanol fermentation. It seems that regarding ethanol fermentation from glucose, ATP is generally regenerated even in the presence of acetic acid without affecting the fermentation ability so much, however, regarding ethanol fermentation from xylose, ATP is poorly regenerated in the presence of acetic acid in that the fermenting ability deteriorates.

Also, while glucose is utilized in the glycolytic system and converted into ethanol, xylose is converted into ethanol via the pentose phosphate pathway and the glycolytic system. It is therefore possible that the pentose phosphate pathway may be affected in some way by acetic acid, however, it has not been yet determined as to what enzyme involved in the pentose phosphate pathway is directly influenced by acetic acid. Accordingly, a strategy for handling weak acids such as acetic acid and formic acid of the fermentation inhibitors has not yet been established.

The inventors have investigated the relation between acetic acid and pH in a fermentation medium using the engineered Saccharomyces cerevisiae MN8140X strain into which the genes for XR, XDH, and XK have been introduced, and found that inhibition of fermentation does not occur in this yeast even in the presence of acetic acid when the pH is adjusted from acidic toward neutral. It has been also reported that the same results are obtained in the engineered yeast into which the genes for XI and XK have been introduced (Non-Patent Document 9).

However, the control of pH is not practical to develop ethanol production from cellulose-based biomass to an industrial scale because it is costly and the contamination with other microorganisms may occur with around neutral pH. Accordingly, efficient ethanol fermentation from xylose in the presence of acetic acid (at acidic pH) is desired.

As described above, research has been extensively carried out in an attempt to achieve efficient ethanol fermentation from xylose even in the presence of a fermentation inhibitor such as acetic acid. However, there is absolutely no successful case of providing a yeast with a tolerance to a fermentation inhibitor or achieving efficient ethanol fermentation from xylose in the presence of the fermentation inhibitor.

Now then, it has been reported that the activities of transaldolase (TAL) and transketolase (TKL), which are involved in the pentose phosphate pathway (FIG. 1), relate to the rate of xylose utilization (Non-Patent Documents 10 and 11). It has been also reported that the gene for TAL1 derived from the yeast Pichia stipitis is overexpressed in the yeast Saccharomyces cerevisiae to facilitate ethanol fermentation (Non-Patent Document 12).

However, there remains unclear as to the relation between the overexpression of TAL and TKL and the tolerance to a fermentation inhibitor such as acetic acid in yeast.

### PRIOR ART DOCUMENTS

### Non-Patent Documents

Non-Patent Document 1: B. C. H. Chu and H. Lee, “Genetic improvement of Saccharomyces cerevisiae for xylose fermentation”, Biotechnology Advances, 2007, vol. 25, pp. 425-441

Non-Patent Document 2: C. Lu and T. Jeffries, “Shuffling of promoters for multiple genes to optimize xylose fermentation in an engineered Saccharomyces cerevisiae strain”, Appl. Environ. Microbiol., 2007, vol. 73, pp. 6072-6077

Non-Patent Document 3: M. Kuyper et al., “Metabolic engineering of a xylose-isomerase-expressing Saccharomyces cerevisiae strain for rapid anaerobic xylose fermentation”, FEMS Yeast Res., 2005, vol. 5, pp. 399-409

Non-Patent Document 4: J. R. M Almeida et al., “Increased tolerance and conversion of inhibitors in lignocellulosic hydrolysates by Saccharomyces cerevisiae”, J. Chem. Technol. Biotechnol., 2007, vol. 82, pp. 340-349

Non-Patent Document 5: A. J. A. van Maris et at, “Alcoholic fermentation of carbon sources in biomass hydrolysates by Saccharomyces cerevisiae: current status”, Antonie van Leeusenhoek, 2006, vol. 90, pp. 391-418

Non-Patent Document 6: E. Palmqvis and B. Hahn-Hagerdal, “Fermentation of lignocellulosic hydrolysates. II: inhibitors and mechanisms of inhibition”, Bioresource Technology, 2000, vol. 74, pp. 25-33

Non-Patent Document 7: S. W. Gorsich et al., “Tolerance to furfural-induced stress is associated with pentose phosphate pathway genes ZWF1, GND1, RPE1, and TKL1 in Saccharomyces cerevisiae”, Appl. Microbiol. Biotechnol., 2006, vol. 71, pp. 339-349

Non-Patent Document 8: A. Petersson et al., “A 5-hydroxymethyl furfural reducing enzyme encoded by the Saccharomyces cerevisiae ADH6 gene conveys HMF tolerance”, Yeast, 2006, vol. 23, pp. 455-464

Non-Patent Document 9: E. Bellissimi et at, “Effects of acetic acid on the kinetics of xylose fermentation by an engineered, xylose-isomerase-based Saccharomyces cerevisiae strain”, FEMS Yeast Res., 2009, vol. 9, pp. 358-364

Non-Patent Document 10: M. Walfridsson et al., “Xylose-metabolizing Saccharomyces cerevisiae strains overexpressing the TKL1 and TAL1 genes encoding the pentose phosphate pathway enzymes transketolase and transaldolase”, Appl. Environ. Microbiol., 1995, vol. 61, pp. 4184-4190

Non-Patent Document 11: J. -P. Pitkanen et al., “Xylose chemostat isolates of Saccharomyces cerevisiae show altered metabolite and enzyme levels compared with xylose, glucose, and ethanol metabolism of the original strain”, Appl. Microbiol. Biotechnol., 2005, vol. 67, pp. 827-837

Non-Patent Document 12: Y-S. Jin et al., “Improvement of xylose uptake and ethanol production in recombinant Saccharomyces cerevisiae through an inverse metabolic engineering approach”, Appl. Environ. Microbiol., 2005, vol. 71, pp. 8249-8256

### SUMMARY OF INVENTION

### Problems to be Solved by the Invention

An object of the present invention is to provide a method for producing ethanol efficiently even in the presence of a fermentation inhibitor in a saccharified biomass.

### Means for Solving the Problems

The inventors have conducted diligent research to solve the problem, and then found that a transformed yeast that has been obtained by introducing a gene for a metabolic enzyme involved in the pentose phosphate pathway (hereinafter, to which is also referred as a “pentose phosphate pathway metabolic enzyme”) into a xylose-utilizing yeast and overexpresses the gene is tolerant to a fermentation inhibitor in a saccharified biomass and thus accomplished the present invention.

The present invention provides a method for producing ethanol from biomass, comprising: culturing a transformed xylose-utilizing yeast to overexpress a gene for at least one pentose phosphate pathway metabolic enzyme, with a saccharified biomass.

In one embodiment, the saccharified biomass contains a fermentation inhibitor.

In another embodiment, the fermentation inhibitor is acetic acid or formic acid.

In another embodiment, the pentose phosphate pathway metabolic enzyme is at least one selected from the group consisting of transaldolase and transketolase.

### Effects of Invention

According to the method of the invention, ethanol can be efficiently produced even in the presence of a fermentation inhibitor in a saccharified biomass. It is thus possible to produce bioethanol using lignocellulose-based biomass, such as rice straw, straw, and wood scrap, as a raw material to avoid the competition with food.

### BRIEF DESCRIPTION OF DRAWINGS

FIG. 1 is a schematic diagram showing the pentose phosphate pathway.

FIG. 2 shows graphs indicating a change over time of the concentrations of the substrate (xylose) and products (including ethanol) in the fermentation liquor during the ethanol fermentation by MN8140X strain in the absence of acetic acid (0 mM; (a)) and in the presence of 30 mM (b) and 60 mM (c) of acetic acid.

FIG. 3 shows graphs indicating the accumulated amounts of R5P (a), E4P (b), and S7P (c) accumulated per mg of dry yeast cells during the ethanol fermentation by MN8140X strain in the absence of acetic acid (0 mM) and in the presence of 30 mM and 60 mM of acetic acid.

FIG. 4 shows schematic diagrams indicating the structures of plasmids pGK404-TAL1 (a), pGK404 (b), pGK405-TKL1 (c), and pGK405 (d).

FIG. 5 shows graphs indicating a change over time of the concentrations of the substrate (xylose) and products (including ethanol) in the fermentation liquor during the ethanol fermentation by PGK404/TAL1 strain (TAL1 overexpressing strain) or PGK404 (control) strain in the absence of acetic acid (0 mM; (a)) and in the presence of 30 mM (b) of acetic acid.

FIG. 6 shows graphs indicating a change over time of the concentrations of the substrate (xylose) and products (including ethanol) in the fermentation liquor during the ethanol fermentation by PGK405/TKL1 strain (TKL1 overexpressing strain) or PGK405 (control) strain in the absence of acetic acid (0 mM; (a)) and in the presence of 30 mM (b) of acetic acid.

FIG. 7 shows graphs indicating a change over time of the concentrations of the substrate (xylose) and products (including ethanol) in the fermentation liquor during the ethanol fermentation by PGK404/TAL1 strain (TAL1 overexpressing strain) (a, b) or PGK404 (control) strain (c, d) in the presence of 15 mM (a, c) and 30 mM (b, d) of formic acid.

### MODE FOR CARRYING OUT THE INVENTION

Biomass refers to carbohydrate materials derived from biological resources, including starch derived from corn and the like, and molasses (blackstrap molasses) derived from sugar cane or the like, and also lignocellulose-based biomass such as wastes generated during processing of biological materials such as rice, barley and wheat, corn, sugar cane, and wood (pulp). In the present invention, lignocellulose-based biomass is preferably used to avoid the competition with food, including rice straw, straw, and wood scrap.

Saccharification of biomass refers to degradation of biomass of polysaccharide to monosaccharide, including that the monosaccharide then undergoes overdegradation. (to generate by-products such as acetic acid and formic acid). The processes for saccharification to be employed in the present invention include enzymatic treatment, treatment with diluted sulfuric acid and hydrothermal treatment. In terms of cost, treatment with diluted sulfuric acid and hydrothermal treatment are preferable.

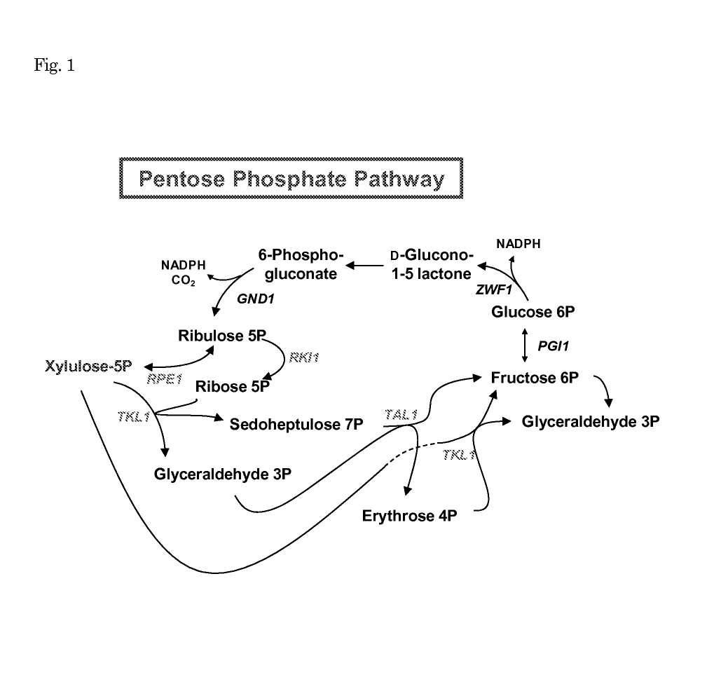

Examples of pentose phosphate pathway metabolic enzymes include transaldolase (TAL), transketolase (TKL), ribose-5-phosphate isomerase (RKI), and ribulose-5-phosphate-3-epimerase (RPE) (see FIG. 1). For example, TAL and TKL are preferable to eliminate the accumulation of ribose-5-phosphate (R5P), erythrose-4-phosphate (E4P), and sedoheptulose-7-phosphate (S7P), which have been found to be significantly accumulated as intermediate metabolites from the metabolism analysis of a xylose-utilizing yeast during ethanol fermentation in the presence of a fermentation inhibitor.

The yeast to be used in the present invention is a transformed xylose-utilizing yeast into which the gene for a pentose phosphate pathway metabolic enzyme has been introduced. The xylose-utilizing yeast to be used for transformation is not particularly limited as long as it is any yeast that can produce ethanol from xylose through ethanol fermentation, including a xylose-utilizing yeast obtained by introducing into the yeast Saccharomyces cerevisiae a plasmid for imparting a xylose-utilizing ability, which can be prepared, for example, as described in S. Katahira et al., Appl. Microbiol. Biotechnol., 2006, vol. 72, pp. 1136-1143.

The process for introducing a gene into a yeast is not particularly limited, including lithium acetate treatment, electroporation, and protoplast. The gene introduced may be present in the form of a plasmid, inserted into the chromosome of yeast, or integrated in the yeast chromosome by homologous recombination.

To introduce the genes for a pentose phosphate pathway metabolic enzyme into a xylose-utilizing yeast, the gene for the metabolic enzyme is preferably inserted into a plasmid. The plasmid preferably contains a selectiable marker and a replication gene for Escherichia coli to facilitate the preparation of a plasmid and detection of a transformant. Examples of selectable markers include drug resistant genes and auxotrophic genes. Examples of drug resistant genes include, but not limited to, ampicillin resistant gene (Amp^{r}) and kanamycin resistant gene (Kan^{r}). Examples of auxotrophic genes include, but not limited to, genes for N-(5′-phosphoribosyl)anthranilate isomerase (TRP1), tryptophan synthase (TRP5), β-isopropylmalate dehydrogenase (LEU2), imidazoleglycerol phosphate dehydrogenase (HIS3), histidinol dehydrogenase (HIS4), dihydroorotic acid dehydrogenase (URA1), and orotidine-5-phosphate decarboxylase (URA3). A replication gene for yeast is not necessarily needed. The plasmid preferably contains a suitable promoter and terminator to express the gene for a pentose phosphate pathway metabolic enzyme in a yeast, including, but not limited to, promoters and terminators of genes for phosphoglycerate kinase (PGK), glyceraldehyde 3′-phosphate dehydrogenase (GAPDH), and glyceraldehyde 3′-phosphate dehydrogenase (GAP). The plasmid preferably contains a gene necessary for homologous recombination, including, but not limited to, Trp1, LEU2, HIS3, and URA3. The plasmid preferably contains a secretion signal sequence as necessary. Examples of the plasmids as described above include pIU-GluRAG-SBA and pIH-GluRAG-SBA as described in R. Yamada et al., Enzyme Microb. Technol., 2009, vol. 44, pp. 344-349. The gene for a pentose phosphate pathway metabolic enzyme is inserted between the promoter and the terminator of such plasmids.

When introducing a plasmid having the gene for a pentose phosphate pathway metabolic enzyme into a xylose-utilizing yeast, it is preferable to cut one location of the plasmid so as to create a linearized form so that such a gene can be integrated into the chromosome by homologous recombination.

The transformed yeast can be prepared in this manner, which overexpresses the gene for a pentose phosphate pathway metabolic enzyme. Overexpression of the gene for a pentose phosphate pathway metabolic enzyme can be verified with the procedure commonly known to those skilled in the art such as RT-PCR.

In the method of the present invention, such a transformed yeast to overexpress the gene for a pentose phosphate pathway metabolic enzyme is cultured with a saccharified biomass. A fermentation inhibitor, such as acetic acid occurring due to overdegradation of biomass, may be present in a saccharified biomass. The transformed yeast to be used in the present invention is tolerant to such a fermentation inhibitor, and proceeds with ethanol fermentation without inhibition to produce ethanol in the medium.

Culturing the transformed yeast can be suitably carried out with the procedure commonly known to those skilled in the art. The pH of the medium is preferably about 4 to about 6, and most preferably about 5. The dissolved oxygen concentration in the medium during aerobic culture is preferably about 0.5 to about 6 ppm, more preferably about 1 to about 4 ppm, and most preferably about 2 ppm. The temperature for culture is about 20 to about 45° C., preferably about 25 to about 35° C., and most preferably about 30° C. It is preferable that the yeast is cultured to 10 g (wet weight)/L or greater, preferably 25 g (wet weight)/L or greater, and more preferably 37.5 g (wet weight)/L or greater of yeast cells, and the culture period is about 20 to about 50 hours. The transformed yeast can be cultured under aerobic conditions prior to fermentation to increase the amount of yeast cells.

### EXAMPLES

The present invention shall be described in detail below by way of examples, but the present invention is not limited to the examples.

### Reference Example 1

### Fermentation Test and Metabolism Analysis for Yeast MN8140X Strain)

(Fermentation Test)

Into both MT8-1 strain (MATa) (obtained from the National Institute of Technology and Evaluation) and NBRC1440 strain (MATα) (obtained from the National Institute of Technology and Evaluation) of Saccharomyces cerevisiae, the plasmid pIUX1X2XK for imparting a xylose-utilizing ability (prepared as described in S. Katahira et al., Appl. Microbiol. Biotechnol., 2006, vol. 72, pp.1136-1143 as the plasmid for coexpressing xylose reductase (XR) and xylitol dehydrogenase (XDH) derived from Pichia stipitis and xylulokinase (XK) derived from Saccharomyces cerevisiae) was introduced by lithium acetate treatment, and the two resulting transformed yeasts were then conjugated by mating to obtain a diploid transformed yeast MN8140X strain. This xylose-utilizing yeast MN8140X strain was used to carry out ethanol fermentation from xylose.

The influence of acetic acid was investigated as the condition for fermentation. To YP medium (10 g/L of yeast extract, 20 g/L of Bacto Peptone) with xylose at the initial concentration of 40 g/L and yeast cells at 2.5 g/L (wet weight), no acetic acid was added (0 mM) or acetic acid was added at the concentration of 30 mM or 60 mM, and the yeast was then cultured.

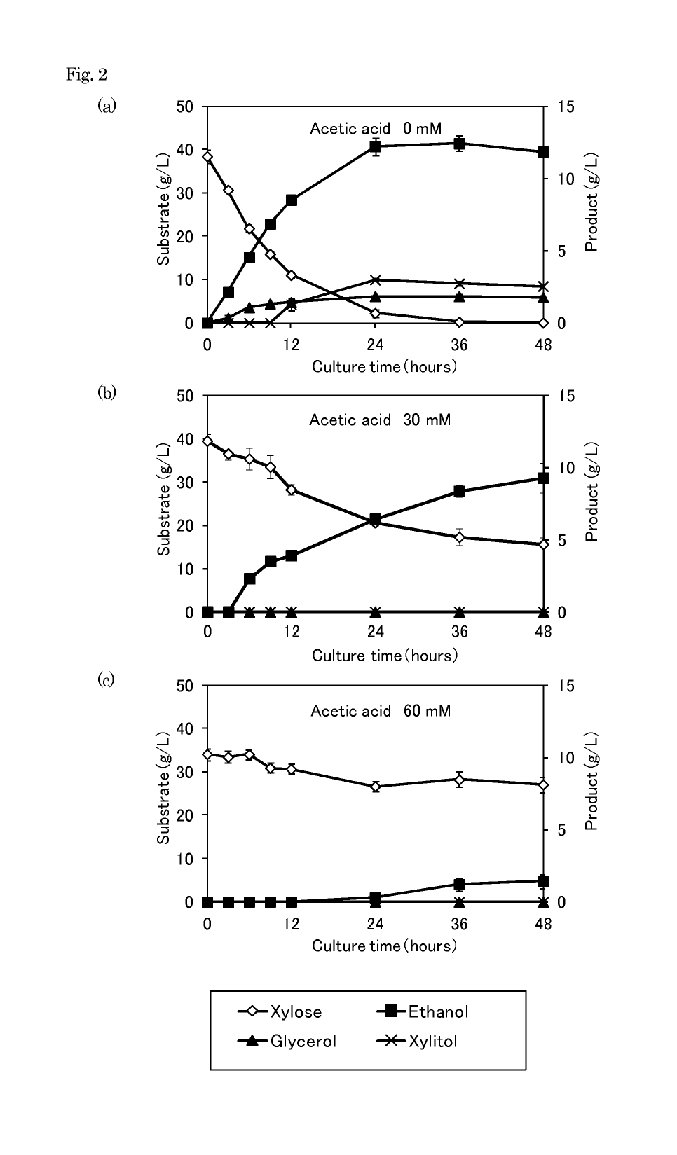

The amounts of xylose and products, including ethanol, in the medium were determined over time by HPLC (High performance liquid chromatography system; manufactured by Shimadzu Corporation) using Shim-pack SPR-Pb (manufactured by Shimadzu Corporation) as a separation column, ultrapure water (purified water Milli-Q manufactured by Nihon Millipore K.K.) as a mobile phase, and a refractive index detector as a Detector under the conditions of the flow rate of 0.6 mL/min and the temperature of 80° C. The results are shown in FIG. 2.

As is clear from FIG. 2, with increasing concentration of acetic acid, the rate of xylose consumption was decreased, and accordingly both the rate and amount of ethanol production were decreased. From this, it can be understood that ethanol fermentation from xylose is greatly inhibited due to the presence of acetic acid.

(Metabolism Analysis)

For the respective acetic acid concentrations, yeast cells (in 5 mL of medium) were collected after 4 hours, 6 hours, and 24 hours of culture, washed twice with distilled water, and then freeze-dried. Ten mg of the resulting dried cells were placed in a tube for disruption together with 300 μg of glass beads (0.5 mm in diameter, manufactured by Yasui Kikai Corporation), 500 μL of methanol, 180 μL of ultrapure water, and 20 μL of piperazine-1,4-bis(2-ethanesulfonic acid) (PIPES) buffer, and disrupted with Multi-beads shocker (manufactured by Yasui Kikai Corporation) in 5 disruption cycles of disrupting at 2,500 rpm for 60 seconds at 4° C. and cooling for 60 seconds. Subsequently, the tube for disruption was shaken at 1200 rpm for 30 minutes at 4° C. and then centrifuged at 15,000 rpm for 3 minutes at 4° C., and 630 μL of supernatant was transferred to a 15 mL microtube. To this, ultrapure water (270 μL) was added and mixed. Subsequently, the microtube was centrifuged at 15,000 rpm for 3 minutes at 4° C., and 300 μL of supernatant (yeast extract) was transferred to another 1.5 mL microtube. This yeast extract was dried, and 10 μL of ultrapure water was added to the residue to obtain a concentrated yeast extract.

This concentrated yeast extract was qualitatively and quantitatively analyzed with CE-TOFMS (Capillary Electrophoresis Time-Of-Flight Mass Spectrometer, manufactured by Agilent Technologies, Inc.) of electrophoresis using Fused Silica Capillary (50 μm i.d., total length 100 cm) as an electrophoresis capillary and 30 mM of ammonium formate (pH 10) as an electrophoresis buffer under the conditions of the voltage of 30 kV and the temperature of 20° C., and mass spectrometry in ESI-Negative under the conditions of the flow rate for sheath fluid (50% methanol) of 8 μL/min, the capillary voltage of 3.5 kV, the fragment voltage of 100 V, and the flow rate for dry gas of 10 L/min (300° C.), employing Mass Hunter software.

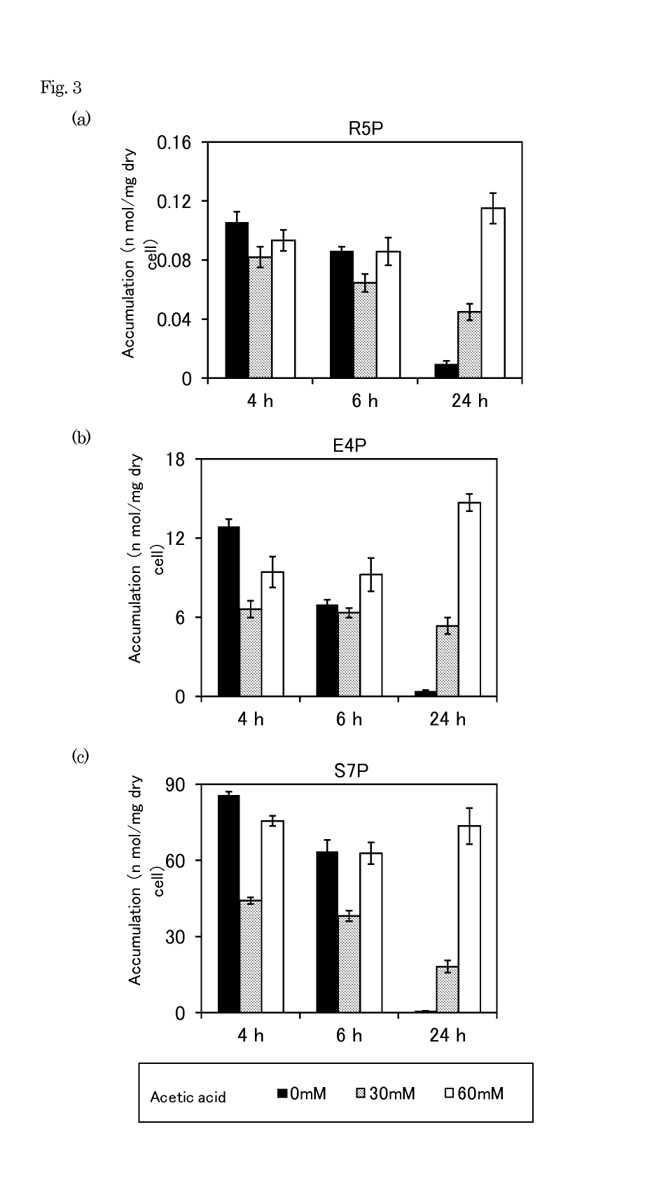

The yeast extract was analyzed with CE-TOFMS as mentioned above to identify the components therein, and the amounts of the respective components were determined on the basis of the peak areas for the components in the mass chromatogram. Calibration curves were created using standard samples for intermediate metabolites, ribose-5-phosphate (R5P), erythrose-4-phosphate (E4P), and sedoheptulose-7-phosphate (S7P), of the pentose phosphate pathway with PIPES as an internal standard, and were used to determine the amount. FIG. 3 shows the accumulated amounts of R5P, E4P, and S7P in the yeast cells.

As is clear from FIG. 3, with increasing concentration of acetic acid, the accumulated amounts of R5P, E4P, and S7P were increased.

### Example 1

### Preparation of Plasmid for Overexpression of TAL1 or TKL1

In an attempt to avoid the accumulation of R5P, E4P, or S7P, a plasmid was constructed for overexpression of the gene for transaldolase (TAL) or transketolase (TKL), which is the enzyme considered to be involved in the metabolism thereof.

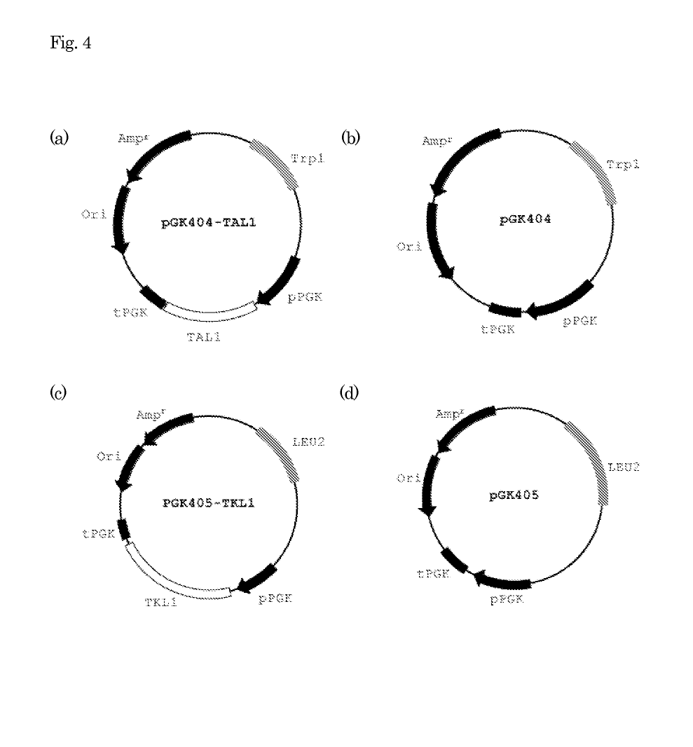

A plasmid pGK404-TAL1 (FIG. 4(a)) was prepared by inserting Saccharomyces cerevisiae TAL1 gene (SEQ ID NO. 1) between the promoter and the terminator of a plasmid pGK404 (FIG. 4(b); prepared as described in J. Ishii et al., J. Biochem., 2009, vol. 145, pp. 701-708), which has a PGK promoter and a PGK terminator. The TAL1 gene used for insertion was prepared by preparing a DNA fragment by PCR as commonly conducted using primers ScTAL-SpeI-F (SEQ ID NO. 3) and ScTAL-BamHI-R (SEQ ID NO. 4) with as a template a genomic DNA extracted from Saccharomyces cerevisiae MT8-1 strain (MATa) according to the commonly used procedure, and treating this fragment with restriction enzymes SpeI and BamHI. The resulting plasmid pGK404-TAL1 contained an Amp^{r} gene to provide an ampicillin resistance with the transformant and a yeast-derived Trp1 gene necessary for homologous recombination.

Similarly, a plasmid pGK405-TKL1 (FIG. 4(c)) was prepared by inserting Saccharomyces cerevisiae TKL1 gene (SEQ ID NO. 5) between the promoter and the terminator of a plasmid pGK405 (FIG. 4(d); prepared as described in J. Ishii et al., J. Biochem., 2009, vol. 145, pp. 701-708), which has a PGK promoter and a PGK terminator. The TKL1 gene used for insertion was prepared by preparing a DNA fragment by PCR as commonly conducted using primers ScTKL-SalI-F (SEQ ID NO. 7) and ScTKL-SpeI-R (SEQ ID NO. 8) with as a template a genomic DNA extracted from Saccharomyces cerevisiae MT8-1 strain (MATa) according to the commonly used procedure, and treating this fragment with restriction enzymes SalI and SpeI. The resulting plasmid pGK405-TKL1 contained an Ampr gene to provide an ampicillin resistance with the transformant and a yeast-derived LEU2 gene necessary for homologous recombination.

### Example 2

### Preparation of TAL1 or TKL1 Overexpressing Strain

The plasmid pGK404-TAL1 or pGK404 prepared in Example 1 was treated with a restriction enzyme EcoRV to cleave Trp1 gene into a linearized form.

The plasmid pGK405-TKL1 or pGK405 prepared in Example 1 was treated with a restriction enzyme EcoRV to cleave LEU2 gene into a linearized form.

Into a transformant obtained by introducing a plasmid pIUX1X2XK into Saccharomyces cerevisiae MT8-1 strain (MATa), the linearized plasmid was introduced by lithium acetate treatment to obtain the strains: MT8-1/pIUX1X2XK/pGK404-TAL1 (PGK404/TAL1 strain), MT8-1/pIUX1X2XK/pGK404 (PGK404 (control) strain), MT8-1/pIUX1X2XK/pGK405-TKL1 (PGK405/TKL1 strain), and MT8-1/pIUX1X2XK/pGK405 strain (PGK405 (control) strain). The PGK404/TAL1 strain and the PGK404 (control) strain were cultured in SD-UW solid medium (6.7 g/L of Yeast Nitrogen Base without Amino Acids [manufactured by Difco], 20 g/L of glucose, 0.02 g/L of uracil, 0.02 g/L of tryptophan), and the PGK405/TKL1 strain and the PGK405 (control) strain were cultured in SD-LU solid medium (6.7 g/L of Yeast Nitrogen Base without Amino Acids [manufactured by Difco], 20 g/L of glucose, 0.1 g/L of leucine, 0.02 g/L of uracil).

### Example 3

### Measurement of Enzyme Activity for PGK404/TAL1 Strain or PGK405/TKL1 Strain

The enzyme activity was measured for the PGK404/TAL1 strain, the PGK404 (control) strain, the PGK405/TKL1 strain, or the PGK405 (control) strain prepared in Example 2.

For each strain, yeast cells were aerobically cultured in YPD medium (10 g/L of yeast extract, 20 g/L of Bacto Peptone, 20 g/L of glucose) to reach the stationary phase and then centrifuged at 5000 g for 5 minutes at 4° C. After removal of the supernatant, 10 mM of potassium phosphate buffer (pH 7.5) and 2 mM of EDTA were added to the cell-containing precipitate and mixed. Subsequently, the mixture was centrifuged at 5000 g for 5 minutes at 4° C., and 100 mM of potassium phosphate buffer (pH 7.5), 2 mM of magnesium chloride, and 2 mM of dithiothreitol were then added to the cell-containing precipitate and mixed. Glass beads (0.5 mm in diameter, manufactured by Yasui Kikai Corporation) were further mixed, and the cells were disrupted with Multi-beads shocker (manufactured by Yasui Kikai Corporation) at 2500 rpm for 5 minutes at 4° C. Subsequently, the tube for disruption containing the cells was centrifuged at 30000 g for 30 minutes at 4° C., and 300 μL of supernatant (yeast extract) was collected. The TAL activity and the TKL activity were measured for this yeast extract.

The TAL activity was determined by an oxide generated by reacting NADH with TAL (transaldolase) to generate oxides and measuring the generated oxides at the absorbance of 340 nm according to the procedure in “Methods of Enzymatic Analysis”, edited by H.-U. Bergmeyer, Academic Press, New York, N.Y. 1974. The TKL activity was determined with 100 mM of triethanolamine buffer (pH 7.8) according to the procedure in P. M. Bruinenberg et al., “An enzymatic analysis of NADPH production and consumption in Candida utilis”, J. Gen. Microbiol., 1983, vol. 129, pp. 965-971. The concentration of protein was determined with a protein assay kit manufactured by Bio-Rad Laboratories. The results are shown in Table 1.

TABLE 1

Enzyme activity (U/mg protein)

| Strain | TAL | TKL |
|---|---|---|
| PGK404/TAL1 | 0.111 ± 0.009 | 0.066 ± 0.021 |
| PGK404 (control) | 0.054 ± 0.004 | 0.052 ± 0.016 |
| PGK405/TKL1 | 0.027 ± 0.011 | 0.149 ± 0.009 |
| PGK405 (control) | 0.021 ± 0.012 | 0.048 ± 0.013 |

Values are presented as the average ± S.D. (n = 3).

As is clear from Table 1, the PGK404/TAL1 strain had a TAL activity about 2.1 times greater than that of the PGK404 (control) strain, and the PGK405/TAL1 strain had a TKL activity about 3.1 times greater than that of the PGK405 (control) strain. Accordingly, it can be understood that the PGK404/TAL1 strain has an enhanced TAL1 activity (TAL1 overexpressing strain), and the PGK405/TAL1 strain has an enhanced TKL activity (TKL1 overexpressing strain).

### Example 4

### Metabolism Analysis for TAL1 Overexpressing Strain

The influence of acetic acid was investigated as the condition for fermentation. To YP medium (10 g/L of yeast extract, 20 g/L of Bacto Peptone) with xylose at the initial concentration of 40 g/L and yeast cells at 2.5 g/L (wet weight), no acetic acid was added (0 mM) or acetic acid was added at the concentration of 60 mM, and the yeast was then cultured.

Yeast cells (in 5 mL of medium) were collected after 24 hours of culture, and poured into a polypropylene tube containing 7 mL of methanol that had previously been cooled in a −40° C. cooling bath. This suspension was centrifuged at 5000 g for 5 minutes at −20° C. After removal of the supernatant, 7.5 μL of 1 mM piperazine-1,4-bis(2-ethanesulfonic acid) (PIPES) and 7.5 μL of 100 mM adipic acid were added to the cell-containing precipitate, and 75% (v/v) ethanol that had previously been boiled at 95° C. was further added and mixed using a vortex mixer. Subsequently, the mixture was thermally treated for 3 minutes at 95° C. and centrifuged at 15000 rpm for 5 minutes at 4° C., and 300 μL of supernatant (yeast extract) was transferred to a 1.5 mL microtube. The yeast extract was dried, and 10 μL of ultrapure water was added to the residue to obtain a concentrated yeast extract.

This concentrated yeast extract was qualitatively and quantitatively analyzed with CE-TOFMS (Capillary Electrophoresis Time-Of-Flight Mass Spectrometer, manufactured by Agilent Technologies, Inc.) of electrophoresis using Fused Silica Capillary (50 μm i.d., total length 100 cm) as an electrophoresis capillary and 30 mM of ammonium formate (pH 10) as an electrophoresis buffer under the conditions of the voltage of 30 kV and the temperature of 20° C., and mass spectrometry in ESI-Negative under the conditions of the flow rate for sheath fluid (50% methanol) of 8 μL/min, the capillary voltage of 3.5 kV, the fragment voltage of 100 V, and the flow rate for dry gas of 10 L/min (300° C.), employing Mass Hunter software.

The yeast extract was analyzed with CE-TOFMS as mentioned above to identify the components therein, and the amounts of the respective components were determined on the basis of the peak areas for the components in the mass chromatogram. Calibration curves were created using standard samples for intermediate metabolites, 6-phosphogluconate (6PG), ribose-5-phosphate (R5P), ribulose-5-phosphate (Ru5P), and sedoheptulose-7-phosphate (S7P), of the pentose phosphate pathway with PIPES as an internal standard, and were used to determine the amount. Table 2 shows the accumulated amounts of 6PG, R5P, Ru5P, and S7P in the yeast cells.

TABLE 2
Accumulated amount (nmol/g dry weight)

| | PGK404/TAL1 | | PGK404 (control) | |
|---|---|---|---|---|
| Metabolite | 0 mM Acetic acid | 60 mM Acetic acid | 0 mM Acetic acid | 60 mM Acetic acid |
| 6PG | 9.1 ± 0.2 | 21.4 ± 0.5 | 10.9 ± 1.51 | 33.4 ± 3.67 |
| R5P | 53.8 ± 11.8 | 79.1 ± 14.5 | 30.2 ± 2.40 | 50.0 ± 6.63 |
| Ru5P | 4.8 ± 0.7 | 39.7 ± 10.1 | 28.6 ± 5.52 | 50.1 ± 9.32 |
| S7P | 175.8 ± 1.0 | 231.5 ± 6.2 | 812.3 ± 66.78 | 4591.6 ± 256.32 |

Values are presented as the average ± S.D. (n = 4).

As is clear from Table 2, overexpression of TAL1 removed accumulation of intermediate products of the pentose phosphate cycle.

### Example 5

### Fermentation Test in the Presence of Acetic Acid for TAL1 or TKL1 Overexpressing Strain

Ethanol fermentation from xylose in the presence of acetic acid was carried out with the TAL1 overexpressing strain, the control strain for the TAL1 overexpressing strain, the TKL1 overexpressing strain, or the control strain for the TKL1 overexpressing strain prepared in Example 2.

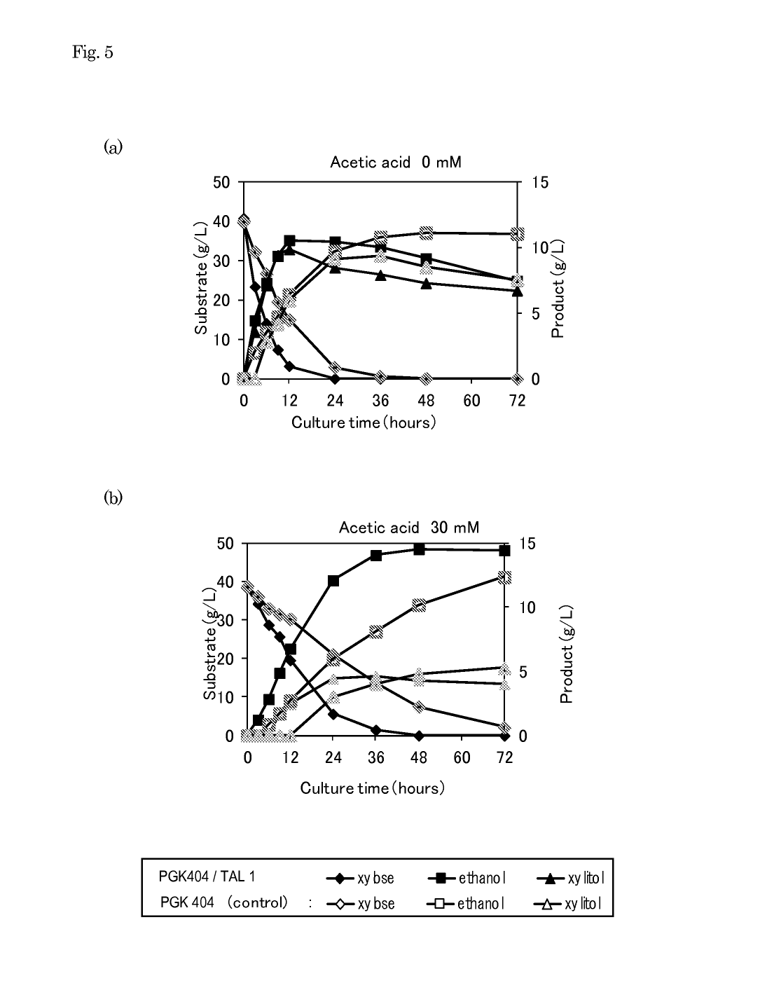

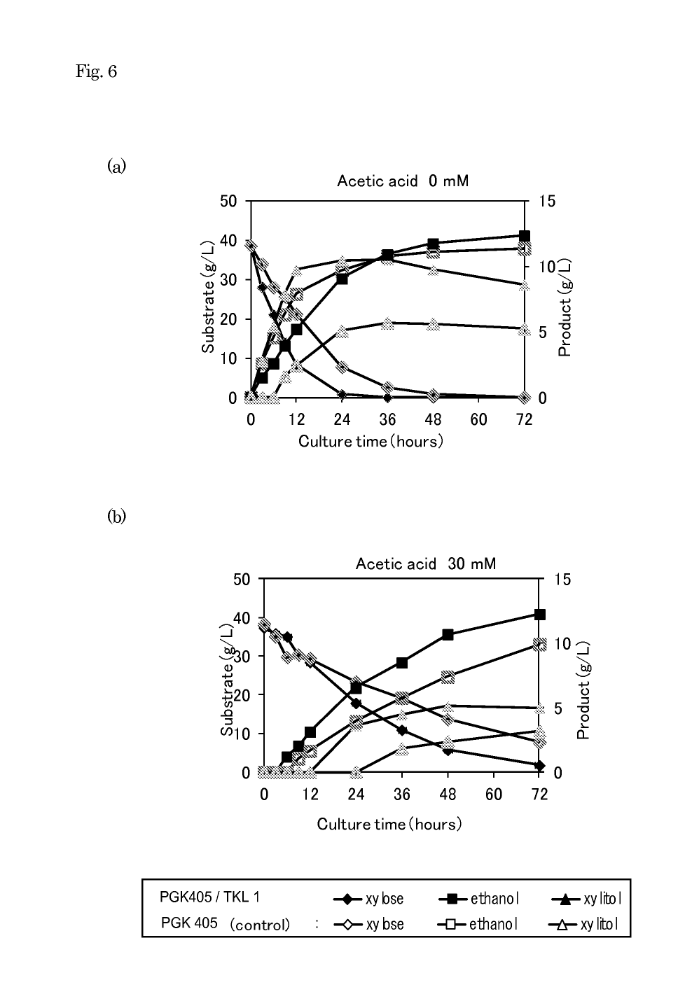

The influence of acetic acid was investigated on the fermentation conditions. To YP medium with xylose at the initial concentration of 40 g/L and yeast cells at 2.5 g/L (wet weight), no acetic acid was added (0 mM) or acetic acid was added at the concentration of 30 mM or 60 mM, and the yeast was then cultured. Determination of the amounts of xylose and products including ethanol in the medium was carried out in the same manner as in Reference Example 1. The results are shown in FIGS. 5 and 6.

As is clear from FIG. 5, regarding the TAL1 overexpressing strain compared with the control strain, in the absence of acetic acid, the rate of xylose consumption was increased, but the final amount of ethanol produced was not varied; on the other hand, in the presence of 30 mM acetic acid, the rate of xylose consumption, the rate of ethanol production, and the final amount of ethanol produced were significantly increased, the ethanol production rate up to 24 hours after the beginning of culture was increased to about two-fold, and the final amount of ethanol produced was increased to about 1.2 fold. The ethanol yield of the TAL1 overexpressing strain in the presence of 30 mM acetic acid was observed to be about 80% of the theoretical yield and far exceed those reported heretofore.

As is clear from FIG. 6, regarding the TKL1 overexpressing strain compared with the control strain, in the absence of acetic acid, the rate of xylose consumption was increased, but the finally produced ethanol amount was not varied; on the other hand, in the presence of 30 mM acetic acid, the rate of xylose consumption, the rate of ethanol production, and the final amount of ethanol produced were significantly increased, the ethanol production rate up to 24 hours after the beginning of culture was increased to about 1.7 fold, and the final amount of ethanol produced was increased to about 1.2 fold.

### Example 6

### Fermentation Test in the Presence of Formic Acid for TAL1 Overexpressing Strain

Ethanol fermentation from xylose in the presence of formic acid was carried out using the TAL1 overexpressing strain, the control strain for the TAL1 overexpressing strain prepared in Example 2.

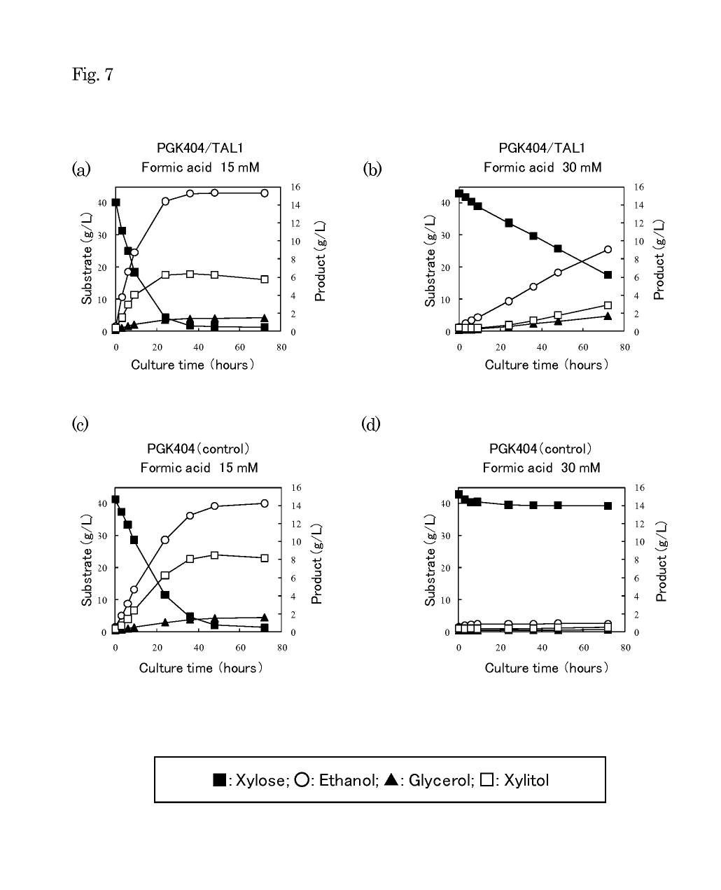

The influence of formic acid was investigated on the fermentation conditions. To YP medium with xylose at the initial concentration of 40 g/L and yeast cells at 2.5 g/L (wet weight), formic acid was added at the concentration of 15 mM or 30 mM, and the yeast was then cultured. Determination of the amounts of xylose and products, including ethanol, in the medium was carried out in the same manner as in Reference Example 1. The results are shown in FIG. 7.

As is clear from FIG. 7, overexpression of TAL1 enhanced the ability of yeast to produce ethanol in the presence of formic acid.

### INDUSTRIAL APPLICABILITY

According to the method of the present invention, ethanol can be efficiently produced even in the presence of a fermentation inhibitor in a saccharified biomass. Accordingly, it is possible to produce bioethanol using lignocellulose-based biomass, such as rice straw, straw, and wood scrap, as a raw material to avoid the competition with food, leading to provision of alternatives to fossil fuels, as well as prevention of global warming and solution of food issues.

1-6. (canceled)

7. A method for producing a xylose-utilizing yeast tolerant to acetic acid and formic acid on culturing it with a saccharified biomass, comprising:
transforming a xylose-utilizing yeast to overexpress a gene for at least one of selected from the group consisting of pentose phosphate pathway metabolic enzymes of transaldolase and transketolase.

8. The method according to claim 7, wherein the tolerance to acetic acid is a tolerance to 30 mM or greater of acetic acid, and the tolerance to formic acid is a tolerance to 15 mM or greater of formic acid.

9. A method for producing ethanol, comprising:
culturing a yeast produced by the method according to claim 7 with a saccharified biomass containing at least acetic acid or formic acid as a fermentation inhibitor.

10. A method for producing ethanol, comprising:
culturing a yeast produced by the method according to claim 8 with a saccharified biomass containing at least acetic acid or formic acid as a fermentation inhibitor.

11. A method for producing a transformed xylose-utilizing yeast tolerant to acetic acid and formic acid, comprising:
transforming a yeast by an expression vector comprising a xylose reductase gene, an expression vector comprising a xylitol dehydrogenase gene, an expression vector comprising a xylulokinase gene and an expression vector comprising a gene selected from the group consisting of a transaldolase gene and a transketolase gene
wherein ethanol fermentation by the transformed xylose-utilizing yeast is not inhibited by a fermentation inhibitor on culturing the transformed xylose-utilizing yeast with saccharified biomass containing the fermentation inhibitor, and
wherein the fermentation inhibitor is acetic acid or formic acid.
